(19) United States
(12) Patent Application Publication
(10) Pub. No.: US 2026/0188476 A1
(43) Pub. Date: Jul. 2, 2026

(54) METHODS AND APPARATUS FOR A TELECARE COMMUNICATION SYSTEM

(71) Applicant: TeleSpecialists, LLC, Fort Myers, FL (US)

(72) Inventors: Muhammad MASUD, Knoxville, TN (US); Nima Mowzoon, Fort Myers, FL (US); Murat Erkoc, Hialeah, FL (US)

(73) Assignee: TeleSpecialists, LLC, Fort Myers, FL (US)

(21) Appl. No.: 19/550,045

(22) Filed: Feb. 25, 2026

Related U.S. Application Data
(63) Continuation of application No. 17/476,883, filed on Sep. 16, 2021, now abandoned.
(60) Provisional application No. 63/130,046, filed on Dec. 23, 2020.

(51) Int. Cl. G16H 40/20; G06Q 10/0631; G16H 40/67

(52) U.S. Cl. CPC G16H 40/20; G06Q 10/063112; G16H 40/67

(57) ABSTRACT

This application relates to apparatus and methods for automatically determining medical professional assignments to provide medical care. In some examples, a computing device receives a case alert for a case for a facility, and determines a plurality of physicians credentialed at the facility. The computing device determines an availability status of each of the physicians based on an availability schedule, and further determines a credentialing index for each of the physicians. Each credentialing index can be based on a forecasted demand of each of a plurality of facilities that each of the physicians is credentialed for. The computing device determines one of the physicians based on the availability status and the credentialing index of each of the plurality of physicians. The computing device then generates a case request for the case for the first physician, and transmits the case request for assignment.

### CROSS-REFERENCE TO RELATED APPLICATIONS

This application is a continuation of co-pending U.S. Utility patent application Ser. No. 17/476,883, filed Sep. 16, 2021, entitled “METHODS AND APPARATUS FOR A TELECARE COMMUNICATION SYSTEM,” which claims priority to U.S. Provisional Patent Application No. 63/130,046 , filed on Dec. 23, 2020 and entitled “METHODS AND APPARATUS FOR A TELECARE COMMUNICATION SYSTEM,” each of which is hereby incorporated by reference in its entirety.

### FIELD OF THE INVENTION

The disclosure relates generally to communication systems and, more particularly, to a telecare communication system.

### BACKGROUND

Patients often are in need of medical attention for a variety of clinical conditions, such as heart attacks and strokes. Medical facilities, such as hospitals, may experience difficulties in delivering timely care due to challenges with staffing, such as a limited number of specialists available to treat a given condition. In some instances, medical facilities may experience an increased number of patients. If the medical facility is not prepared to handle the increased numbers of patient, the patients may also experience a delay in care. As such, there are opportunities to address shortfalls, such as supply and demand shortfalls, with the availability of healthcare professionals and the timeliness of healthcare.

### SUMMARY

In some embodiments, a system includes a database and a first computing device communicatively coupled to the database. The first computing device is configured to receive a case alert for a case for a facility, and determine a plurality of physicians credentialed at the facility based on physician data stored in the database. The first computing device is also configured to determine an availability status of each of the plurality of physicians based on an availability schedule stored in the database. Further, the first computing device is configured to determine a credentialing index for each of the plurality of physicians, where each credentialing index is based on a forecasted demand of each of a plurality of facilities that each of the plurality of physicians is credentialed for. The first computing device is also configured to determine a first physician of the plurality of physicians based on the availability status and the credentialing index of each of the plurality of physicians. The first computing device is further configured to generate a first case request for the case for the first physician, and transmit the first case request to a second computing device.

In some embodiments, a method by a first computing device includes receiving a case alert for a case for a facility, and determining a plurality of physicians credentialed at the facility based on physician data stored in a database. The method also includes determining an availability status of each of the plurality of physicians based on an availability schedule stored in the database. Further, the method includes determining a credentialing index for each of the plurality of physicians, wherein each credentialing index is based on a forecasted demand of each of a plurality of facilities that each of the plurality of physicians is credentialed for. The method also includes determining a first physician of the plurality of physicians based on the availability status and the credentialing index of each of the plurality of physicians. The method further includes generating a first case request for the case for the first physician, and transmitting the first case request to a second computing device.

In some embodiments, a non-transitory computer readable medium having instructions stored thereon, wherein the instructions, when executed by at least one processor, cause a first computing device to perform operations. The operations include receiving a case alert for a case for a facility, and determining a plurality of physicians credentialed at the facility based on physician data stored in a database. The operations also include determining an availability status of each of the plurality of physicians based on an availability schedule stored in the database. Further, the operations include determining a credentialing index for each of the plurality of physicians, wherein each credentialing index is based on a forecasted demand of each of a plurality of facilities that each of the plurality of physicians is credentialed for. The operations also include determining a first physician of the plurality of physicians based on the availability status and the credentialing index of each of the plurality of physicians. The operations further include generating a first case request for the case for the first physician, and transmitting the first case request to a second computing device.

### BRIEF DESCRIPTION OF THE DRAWINGS

The features and advantages of the present disclosures will be more fully disclosed in, or rendered obvious by the following detailed descriptions of example embodiments. The detailed descriptions of the example embodiments are to be considered together with the accompanying drawings wherein like numbers refer to like parts and further wherein:

FIG. 1 is a block diagram of a telecare communication system that includes a telecare computing device in accordance with some embodiments;

FIG. 2 is a block diagram of an exemplary computing device in accordance with some embodiments;

FIG. 3 is a block diagram illustrating various portions of the telecare communication system of FIG. 1 in accordance with some embodiments;

FIG. 4 is a flowchart of an example method that can be carried out by the telecare computing device of FIG. 1 in accordance with some embodiments;

FIG. 5 is a flowchart of another example method that can be carried out by the telecare computing device of FIG. 1 in accordance with some embodiments; and

FIG. 6 is a flowchart of yet another example method that can be carried out by the telecare computing device of FIG. 1 in accordance with some embodiments.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

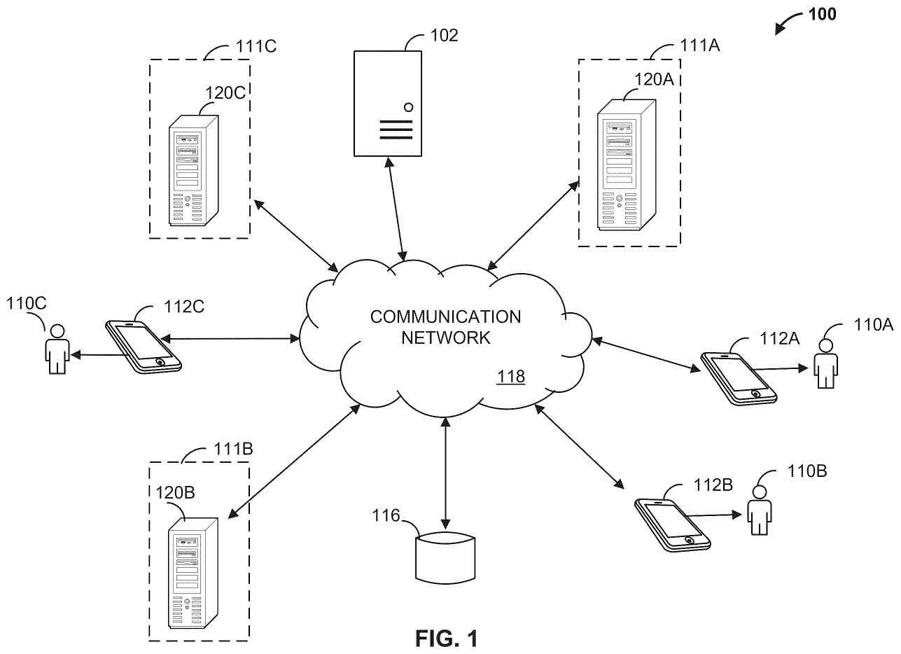

Turning to the drawings, FIG. 1 illustrates a block diagram of a telecare communication system 100 that includes a telecare computing device 102 (e.g., a server, such as a cloud-based server), facility servers 120A, 120B, 120C, physician computing devices 112A, 112B, 112C, and database 116 operatively coupled over communication network 118. Each of telecare computing device 102, facility servers 120A, 120B, 120C, and physician computing devices 112A, 112B, 112C can be any suitable computing device that includes any suitable hardware or hardware and software combination for processing data. For example, each can include one or more processors, one or more field-programmable gate arrays (FPGAs), one or more application-specific integrated circuits (ASICs), one or more state machines, digital circuitry, or any other suitable circuitry. In addition, each can transmit and receive data over communication network 118.

In some examples, each of telecare computing device 102 and facility servers 120A, 120B, 120C can be a computer, a workstation, a laptop, a server such as a cloud-based server, or any other suitable device. Each facility server 120A, 120B, 120C may be associated with (e.g., located within and operated by) a medical facility, such as a hospital. For example, facility server 120A may be associated with medical facility 111A. Similarly, facility server 120B may be associated with medical facility 111B, and facility server 120C may be associated with medical facility 111C.

In some examples, each of the physician computing devices 112A, 112B, 112C can be a cellular phone, a smart phone, a tablet, a personal assistant device, a voice assistant device, a digital assistant, a laptop, a computer, or any other suitable device. Each physician computing device 112A, 112B, 112C may be operated by a medical professional, such as a physician or physician's assistant. For example, physician computing device 112A may be operated by physician 110A, physician computing device 112B may be operated by physician 110B, and physician computing device 112C may be operated by physician 110C.

Although FIG. 1 illustrates three physician computing devices 112A, 112B, 112C, telecare communication system 100 can include any number of physician computing devices 112A, 112B, 112C. Telecare communication system 100 can also include any number of facility servers 120A, 120B, 120C. For example, telecare communication system 100 may include any number of facility servers 120A, 120B, 120C associated with any number of medical facilities 111A, 111B, 111C. Similarly, telecare communication system 100 can include any number of telecare computing devices 102 and databases 116.

Communication network 118 can include one or more communication networks. Each network can be a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. Communication network 118 can provide access to, for example, the Internet.

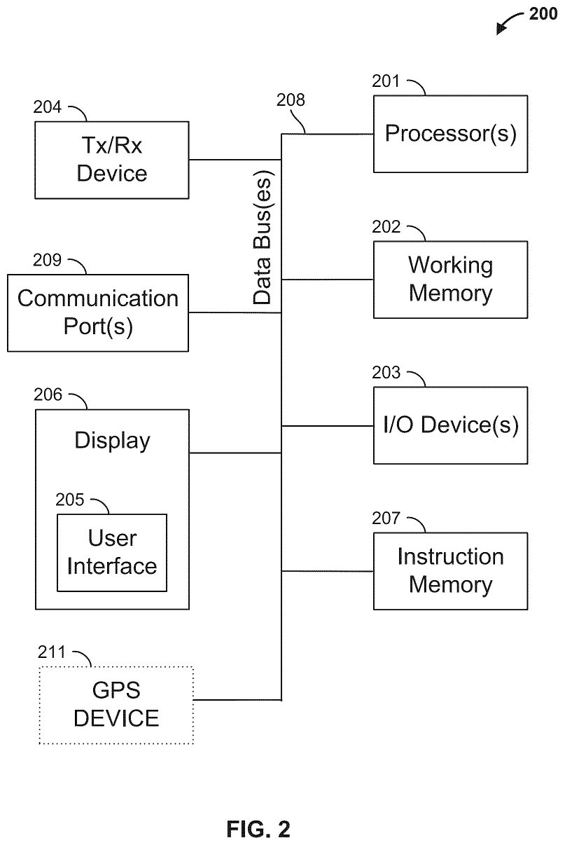

FIG. 2 illustrates an exemplary computing device 200. Computing device 200 may be an example of each of the telecare computing device 102, facility servers 120A, 120B, 120C, and physician computing devices 112A, 112B, 112C of FIG. 1.

As illustrated in FIG. 2, computing device 200 can include one or more processors 201, working memory 202, one or more input/output devices 203, instruction memory 207, a transceiver 204, one or more communication ports 209, a display 206 with a user interface 205, and, optionally, a global positioning system (GPS) device 211, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Instruction memory 207 can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. Processors 201 can be configured to perform a certain function or operation by executing code, stored on instruction memory 207, embodying the function or operation. For example, processors 201 can be configured to execute code stored in instruction memory 207 to perform one or more of any function, method, or operation disclosed herein.

Additionally processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of telecare computing device 102. Working memory 202 can be a random access memory (RAM) such as a static random access memory (SRAM) or dynamic random access memory (DRAM), or any other suitable memory.

Input-output devices 203 can include any suitable device that allows for data input or output. For example, input-output devices 203 can include one or more of a keyboard, a touchpad, a mouse, a stylus, a touchscreen, a physical button, a speaker, a microphone, or any other suitable input or output device.

Communication port(s) 209 can include, for example, a serial port such as a universal asynchronous receiver/transmitter (UART) connection, a Universal Serial Bus (USB) connection, or any other suitable communication port or connection. In some examples, communication port(s) 209 allows for the programming of executable instructions in instruction memory 207. In some examples, communication port(s) 209 allows for the transfer (e.g., uploading or downloading) of data.

Display 206 can be any suitable display, and may display user interface 205. User interfaces 205 can enable user interaction with telecare computing device 102. For example, user interface 205 can be a user interface for an application of a retailer that allows a customer to view and interact with a retailer's website. In some examples, a user can interact with user interface 205 by engaging input-output devices 203. In some examples, display 206 can be a touchscreen, where user interface 205 is displayed on the touchscreen.

Transceiver 204 allows for communication with a network, such as the communication network 118 of FIG. 1. For example, if communication network 118 of FIG. 1 is a cellular network, transceiver 204 is configured to allow communications with the cellular network. In some examples, transceiver 204 is selected based on the type of communication network 118 telecare computing device 102 will be operating in. Processor(s) 201 is operable to receive data from, or send data to, a network, such as communication network 118 of FIG. 1, via transceiver 204.

In some examples, computing device 200 includes GPS device 211. GPS device 211 may be communicatively coupled to the GPS and operable to receive position data from the GPS. For example, GPS device 211 may receive position data identifying a latitude, and longitude, from a satellite of the GPS. Based on the position data, computing device 200 may determine its position and/or a local geographical area (e.g., neighborhood, city, state, etc.).

Referring back to FIG. 1, database 116 can be a remote storage device, such as a cloud-based server, a disk (e.g., a hard disk), a memory device on another application server, a networked computer, or any other suitable remote storage. Although shown remote to telecare computing device 102, in some examples, database 116 can be a local storage device, such as a hard drive, a non-volatile memory, or a USB stick.

Telecare computing device 102 is operable to communicate with database 116 over communication network 118. For example, telecare computing device 102 can store data to, and read data from, database 116. Further, telecare computing device 102 is operable to communicate with facility servers 120A, 120B, 120C and with physician computing devices 112A, 112B, 112C over communication network 118.

Telecare computing device 102 may be operated by a telecare provider, and may allow medical experts, such as physicians 110A, 110B, 110C, to assist with (e.g., provide consult for) medical cases, such as medical cases (e.g., emergencies, such as stroke or heart attack emergencies) at medical or healthcare facilities, such as medical facilities 111A, 111B, 111C. Telecare computing device 102 may provide case intake and dispatch capabilities, physician licensing and credentialing capabilities, quality management capabilities, physician and medical facility capacity management capabilities, physician scheduling capabilities, clinical documentation template capabilities, customizable case reporting capabilities, and may further allow for integration with third-party applications.

For example, telecare computing device 102 may receive case alerts from one or more facility servers 120A, 120B, 120C. The case alert may identify a medical facility 111A, 111B, 111C and a type of alert associated with a case. The type of alert may indicate a medical condition, such as a stroke, for example. When a medical facility 111A, 111B, 111C has a need to request a case, such as for a telehealth consult (e.g., whether for urgent or routine purposes), a facility server 120A, 120B, 120C may transmit a case alert for the case to telecare computing device 102.

In some examples, the medical facility 111A, 111B, 111C calls a call center, and a call center representative provides the relevant data to telecare computing device 102 to generate a case alert. In some examples, telecare computing device 102 is in communication with the call center (e.g., via an API). The call center may identify a number of a calling medical facility 111A, 111B, 111C based on caller ID, and may transmit the identified number to telecare computing device 102. Further, telecare computing device 102 may receive the identified number, and may identify the facility based on searching for the identified number within facility data stored, for example, in database 116. In some examples, telecare computing device 102 includes a web portal whereby the medical facility 111A, 111B, 111C (e.g., via facility server 120A, 120B, 120C) is able to securely submit case alerts for cases (e.g., requests for new consults).

For each case alert, telecare computing device 102 may determine a physician 110A, 110B, 110C to be assigned to the corresponding case. For example, telecare computing device 102 may provide a physician scheduling application that allows for the scheduling of physician shifts. As discussed further below, telecare computing device 102 may determine a physician to assign a case alert based, at least partially on, the scheduling. In some examples, telecare computing device 102 determines the physician based on one or more of physician scheduling, physician credentials, physician status, physician ranking, and/or facility capacities (e.g., facility demand for services).

In some examples, telecare computing device 102 escalates or re-assigns open consults (e.g., assignments) based on designated time intervals to ensure that consults are completed within predetermined time intervals (e.g., designated service level agreements or time targets). In some examples, telecare computing device 102 allows a dispatcher to override or reassign cases as needed. For example, telecare computing device 102 may execute an application that includes an interface (e.g., displayed via display 206) that allows the dispatcher to provide input (e.g. via I/O device 203) to override or reassign cases. Telecare computing device 102 may further include a chat capability (e.g., a chat application) that allows dispatchers to communicate with physicians 110A, 110B, 110C (e.g., via physician computing devices 112A, 112B, 112C) as needed.

Once a physician has been determined, telecare computing device 102 may attempt to assign the case alert to the determined physician 110A, 110B, 110C (e.g., for consultation). For example, telecare computing device 102 may generate and transmit a case request to the physician computing device 112A, 112B, 112C corresponding to the determined physician 110A, 110B, 110C. Reception of the case request may cause the physician computing device 112A, 112B, 112C to execute an application (e.g., mobile application) that allows the determined physician 110A, 110B, 110C to select whether to accept or decline the case. Based on the physician's selection, the application causes the physician computing device 112A, 112B, 112C to generate and transmit a case response indicating the selection to telecare computing device 102.

If the physician 110A, 110B, 110C accepts a case, telecare computing device 102 generates and stores data in database 116 indicating the assignment of the case to the physician 110A, 110B, 110C. Further, telecare computing device 102 may track the progress of the corresponding case until the assigned physician has completed the corresponding consult. If the physician 110A, 110B, 110C declines the case, telecare computing device 102 determines another physician 110A, 110B, 110C to attempt to assign the case to. In some examples, telecare communication system 100 allows for other physicians or specialists to use the platform. For example, other physicians 110A, 110B, 110C 112A, 112B, 112C can communicate with each other in coordinating care for a patient. As an example, telecare communication system 100 may allow neurologists to collaborate with neuro-interventional radiology physicians on coordinating care.

In some examples, the application also allows two-way chat communication with dispatchers operating telecare computing device 102. For example, telecare computing device 102 and the physician computing device 112A, 112B, 112C may exchange chat messages which, in some examples, may be secured (e.g., encrypted and decrypted with public and private keys).

In some examples, telecare computing device 102 provides various clinical documentation templates (e.g., stored in database 116 and provided via user interface 205) to document pertinent details of telehealth consultations for various case types. In some examples, telecare computing device 102 provides note templates (e.g., stored within database 116) whereby physicians enter in consult notes for a case. Telecare computing device 102 may then extract the data from the notes and provide them to various electronic medical record systems (e.g., via a third party application or webpage interfaces).

Further, telecare computing device 102 may determine physicians that are credentialed at a particular medical facility 111A, 111B, 111C, such as the medical facility issuing a case alert. For example, telecare computing device 102 may integrate with medical staff services applications via an Application Programming Interface (API) to track the status of each physician's state license and facility credentialing to ensure that case alerts are assigned only to physicians authorized to work in a particular state and medical facility 111A, 111B, 111C. When a case alert is received, telecare computing device 102 automatically cross references the medical facility 111A, 111B, 111C requesting the consult with available physicians to determine physicians 110A, 110B, 110C that are licensed in the state of the medical facility as well as credentialed within that specific medical facility, thereby allowing only physicians that meet at least both of these criteria to be assigned to the case alert. As a result, telecare computing device 102 may address challenges associated with keeping track of physician scheduling, licensing, and credentialing across multiple facilities and state lines.

Furthermore, telecare computing device 102 may predict medical facility demand and determine capacity needed at each medical facility 111A, 111B, 111C to meet the predicted demand, as well as to meet quality metrics and established service level agreements. For example, telecare computing device 102 may predict demand based on executing decision models that operate on aggregated medical facility historical data for each medical facility 111A, 111B, 111C and, in some examples, aggregated physician historical data for each physician 110A, 110B, 110C. The decision models may determine when and where to license or credential physicians to meet predicted demand, as well as to generate out efficient and cost-effective physician schedules.

In some examples, telecare computing device 102 computes a credentialing index (e.g., credentialing value) for each physician 110A, 110B, 110C. The credentialing index may be based on a forecasted or actual demand of a medical facility 111A, 111B, 111C, a number of medical facilities that a physician is credentialed in, a medical facility's operational performance or efficiency (e.g., hospital workflow factor), and/or the physician's operational performance or efficiency (e.g., physician workflow factor). Telecare computing device 102 may determine physician assignment, physician scheduling, and/or medical facility capacity management decisions based in whole or in part on the credentialing index.

In some examples, telecare computing device 102 allows each medical facility 111A, 111B, 111C to track their own internal billing codes (e.g., via an application) in order to generate invoices for clients (e.g., patients) as well as submit data to a clearinghouse to bill payers for telehealth consults. Further, the application can track medical information including CPT codes, diagnosis information, and other relevant information to generate a claim and bill for the telehealth consult.

In some examples, telecare computing device 102 allows users to obtain a variety of important information such as billing reports, credentialing and licensing reports, physician reports, quality reports and operational reports. Reports can track various key performance indicators and other relevant data to monitor performance, generate billing statements, report on quality outcomes, and to monitor operational effectiveness, for example. Access to the reports can be granted to outside parties, such as healthcare partners receiving telehealth services, in order to allow the outside parties to track their own quality data and improve their operational effectiveness and patient care.

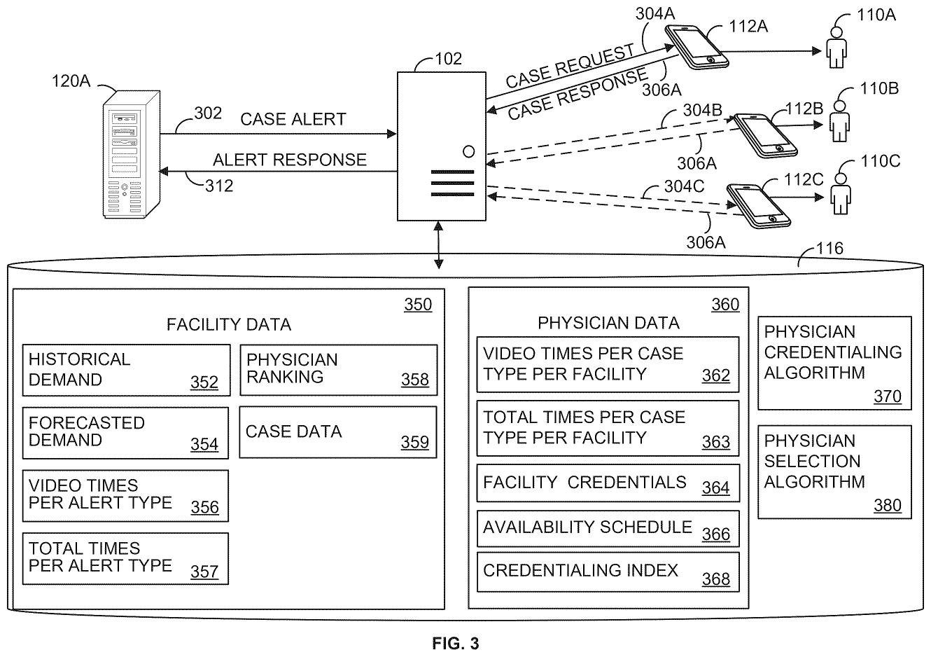

FIG. 3 is a block diagram illustrating examples of various portions of the telecare communication system 100 of FIG. 1. In this example, database 116 stores facility data 350, physician data 360, physician credentialing algorithm 370, and physician selection algorithm 380. Facility data 350 includes data related to each facility (e.g., medical facility 111A, 111B, 111C), such as historical demand 352, forecasted demand 354, video times per alert type 356, physician rankings 358, and case data 359.

Historical demand 352 may identify demand for consults from each facility over a previous period of time, such as a month, a year, five years, etc. Forecasted demand 354 may identify a predicted demand for consults for a facility over a future period of time, such as over the next week, month, year, etc. In some examples, telecare computing device 102 computes forecasted demand 354 based, at least in part, on historical demand 352. In some examples, telecare computing device 102 computes forecasted demand 354 based on one or more forecasting models, such as machine learning based models. Video times per alert type 356 identifies video (e.g., video conference) duration for each physician-patient consultation and aggregated for each type of alert, such as a “stroke” alert or a “heart attack” alert, for the facility, regardless of which physician may have been assigned to a specific consult. Total times per alert type 357 identifies total case handle time by each physician for each case, aggregated for each facility, and identifies a time duration from when a physician accepts a case until a time the physician is complete with the case (e.g., when the physician is in status of “Available” again, or is ready to accept new cases).

Physician rankings 358 identifies a ranking of physicians for each facility, as discussed further below. Case data 359 may identify relevant data and quality metrics that are tracked and aggregated for each case. Case data 359 may include or be based on facility data 350 and/or physician data 360. For example, case data 359 may identify, for each case, the various video times, patient arrival times, and metrics related to drug (e.g., tPA) administration, such as needle times and order times. In addition, case data 359 may also identify quality measures such as door-to-needle (DTN) times or arrival-to-needle (ATN) times, and/or causes of any delays that cause DTN or ATN threshold times to be exceeded. Case data 359 may be used as clinical quality indicators for measuring quality and clinical outcomes of each case and/or, in aggregation, that of a medical facility 111A, 111B, 111C.

Physician data 360 includes data related to each physician, and may include, for example, video times per case type per facility 362, total times per case type per facility 363, facility credentials 364, availability schedule 366, and credentialing index (CI) 368. Video times per case type per facility 362 may identify video times for each physician for each case type (e.g., stroke alerts) per facility. Total times per case type per facility 363 may identify a time duration from when a physician accepts a case until a time the physician is complete with the case, for each case type (e.g., stroke alerts), per facility. Facility credentials 364 may identify which facilities (e.g., of those identified by facility data 350) that each physician is credentialed for (e.g., including facility credentialing and state licensing). Availability schedule 366 identifies, for each physician, their schedule (e.g., work schedule). In some examples, a user may, through a configuration app or webpage (e.g., displayed via display 206), input the schedule of each physician. In some examples, physicians may configure their schedules via an app or web portal hosted by telecare computing device 102. For example, each physician's schedule may identify hours the physician is working and hours the physician is not working, for each day of the week.

Credentialing index 368 identifies a credentialing index for each physician, which may be generated by telecare computing device 102. For example, physician credentialing algorithm 370 identifies and characterizes one or more algorithms to generate credentialing indexes 368 for each physician. Telecare computing device 102 may obtain and execute physician credentialing algorithm 370 to generate the credentialing indexes 368, and is discussed further below.

As indicated in FIG. 3, telecare computing device 102 may receive a case alert 302 from facility server 120A. Facility server 120A may transmit the case alert 302, for example, to request a consult for medical diagnosis or treatment of a medical condition. In response, telecare computing device 102 may determine a first physician to attempt to assign the case alert 302 to. To determine the first physician, telecare computing device 102 may obtain and execute physician selection algorithm 380 from database 116 as described further below.

Once a physician is selected, telecare computing device 102 may generate a case request 304A for the selected physician, and transmit the case request 304A to a physician computing device 112A of the selected physician 110A. The physician may accept or decline the case request 304A (e.g., via an application executed in response to receiving the case request 304A). Physician computing device 112A may generate a case response 306A based on the physician's selection, and may transmit the case response 306A to telecare computing device 102 indicating whether the physician accepts or declines the case.

If the case response 306A indicates that the physician 110A accepts the case, telecare computing device 102 assigns the case to the physician 110A, and may store assignment data in database 116 indicating that physician 110A has been assigned the case. If, however, the case response 306A indicates that the physician 110A declines the case, or if the physician 110A has failed to accept the case within a required response time (e.g., a threshold amount of time, such as 5 minutes), telecare computing device 102 may attempt to assign the case to another physician (e.g., physician 110B, 110C).

For example, telecare computing device 102 may generate a case request 304B for the same case, and transmit case request 304B to physician computing device 112B of physician 110B. Physician 110B may accept or decline the case request 304B. Physician computing device 112B may generate a case response 306B based on the physician's 110B selection, and may transmit the case response 306B to telecare computing device 102 indicating whether the physician 110B accepts or declines the case.

If case response 306B indicates that physician 110B accepts the case, telecare computing device 102 assigns the case to the physician 110B, and may store assignment data in database 116 indicating that physician 110B has been assigned the case. If, however, the case response 306B indicates that the physician 110B declines the case, or if the physician 110B has failed to accept the case within a required response time, telecare computing device 102 may attempt to assign the case to another physician (e.g., physician 110C).

For example, telecare computing device 102 may generate a case request 304C for the same case, and transmit case request 304C to physician computing device 112C of physician 110C. Physician 110C may accept or decline the case request 304C. Physician computing device 112C may generate a case response 306C based on the physician's 110C selection, and may transmit the case response 306C to telecare computing device 102 indicating whether the physician 110C accepts or declines the case.

If case response 306C indicates that physician 110C accepts the case, telecare computing device 102 assigns the case to the physician 110C, and may store assignment data in database 116 indicating that physician 110C has been assigned the case. If, however, the case response 306C indicates that the physician 110C declines the case, or if the physician 110C has failed to accept the case within a required response time, telecare computing device 102 may attempt to assign the case to another physician, or may, in some examples, operate in boost mode, described further below.

In some examples, telecare computing device 102 aggregates case data 359 for each case. In some examples, telecare computing device 102 applies one or more deterministic or stochastic statistical algorithms, adaptive classification models, machine learning algorithms or processes, or artificial neural network models to portions of the case data to determine parameter values characterizing clinical quality indicators (e.g., predetermined clinical quality indicators) for measuring quality and clinical outcomes of each case, physician 110A, and/or medical facility 111A, 111B, 111C. In some examples, telecare computing device 102 can determine trend data characterizing trends of physicians 110A, 110B, 110C and/or medical facilities 111A, 111B, 111C (e.g., by combining trend data for physicians at individual facilities) based on case data 359. Telecare computing device 102 may store the trend data in database 116, and can provide the trend data to physicians 110A, 110B, 110C and medical facilities 111A, 111B, 111C (e.g., via physician computing devices 112A, 112B, 112C, and facility servers 120A, 120B, 120C).

### Credentialing Algorithm

In some examples, telecare computing device 102 may obtain and execute physician credentialing algorithm 370 from database 116 to generate credentialing indexes for one or more physicians. The credentialing indexes may be inputs to the physician selection algorithm 380, as discussed further below.

In some examples, execution of the credentialing algorithm 370 causes telecare computing device 102 to determine credentialing indexes. For example, telecare computing device 102 may obtain a forecasted demand for each facility, such as forecasted demand 354, which may have been precomputed. In some examples, forecasted demand 354 for each facility may be occasionally computed, such as monthly, quarterly, or yearly. In some examples, rather than a forecasted demand, an actual demand, or historical demand 352, may instead be obtained.

In some examples, telecare computing device 102 computes a credential weight of each facility (CWF), which identifies a percent of monthly demand originating from each particular facility (e.g., among all facilities that telecare computing device 102 services). Telecare computing device 102 further computes a raw credential index (RCI) for each physician by summing the CWF corresponding to facilities where each physician is credentialed (e.g., based on facility credentials 364).

Telecare computing device 102 can also compute an average video time for each facility (FV), which is computed based on the video times (e.g., video times per alert type 356) for each alert type (e.g., stroke alerts, heart attach alerts, etc.) served in a facility. Telecare computing device 102 can also compute, for each physician, a physician average video time (PV), which is computed based on the video times of each physician for each facility (e.g., video times per case type per facility 362) for a time period (e.g., the last year, the last month, etc.). In some examples, telecare computing device 102 may compute, for each physician, a physician average total time (PT), which is computed based on total durations each physician spends on each case for each facility (e.g., total times per case type per facility 363) for a time period (e.g., the last year, the last month, etc.).

Telecare computing device 102 can also computes a facility workflow baseline (FWB), which identifies a weighted average of all facility video times (e.g., FV values). Telecare computing device 102 can further compute a facility workflow factor (FWF) for each facility, which is computed in some examples based on dividing the facility's FV by its FWB.

In addition, telecare computing device 102 can compute a time-adjusted forecasted demand (TFD) for each facility. The TFD for each facility is a percent share of each facility's new projected demand, and can be computed in some examples by multiplying the facility's forecasted demand 354 with the corresponding facility workflow factor (FWF).

Telecare computing device 102 may further compute a physician workflow factor (PWF) for each facility. The PWF may be a matrix, and may be computed, in some examples, according to the following. If the physician has PV for a facility, divide each physician average video time (PV) for a facility by the corresponding average video times of the facility (FV). This is the PWF. For facilities in which the physician is credentialed but has no PV, an average of the physician's current PWFs are computed, and the average is used as the new PWF. For example, all current PWFs for facilities in which the physician has a PV are added, and the sum is divided by the total number of those facilities. In some examples, if the average of the current PWFs is zero, the PWF of the physician for the hospital is replaced with a default value, such as 1.

Telecare computing device 102 then computes the Credential Index (CI) for each physician. In some examples, the TFD for each facility is divided by the corresponding PWF of the physician for that facility to determine facility specific CIs. The final CI of the physician is determined based on the facility specific CIs. For example, the final CI of the physician may be determined based on the sum of all the facility specific CIs for the physician. Telecare computing device 102 may store the final CI for each physician in database 116 as CI 368.

### Physician Assignments

Physician selection algorithm 380 identifies and characterizes one or more algorithms to select a physician for the assignment of case alerts. To determine physician assignments for incoming case alerts, telecare computing device 102 may maintain a ranking (e.g., ranked list) of physicians available for case alerts for each medical facility. Telecare computing device 102 may determine the ranking of physicians by applying physician selection algorithm 380 to portions of facility data 350 and/or physician data 360. Telecare computing device 102 may store the physician rankings in database 116 as physician rankings 358.

In some examples, physician selection algorithm 380 is based on one or more rules that are applied to the portions of facility data 350 and/or physician data 360. In one example, telecare computing device 102 generates, for each medical facility 111A, 111B, 111C, an initial ranking of physicians based on their corresponding CI 368. Further, telecare computing device 102 determines which of the ranked physicians to assign to a case alert received from the corresponding medical facility based on each of the ranked physicians'availability schedule 366. For example, telecare computing device 102 may occasionally (e.g., periodically, such as every 15 minutes), update the rankings based on one or more rules. Telecare computing device 102 may apply the rules in a predetermined order.

In some examples, telecare computing device 102 applies one or more of the following rules. Telecare computing device 102 moves a physician with a highest CI, and that has an “Available” status remaining for a minimum period of time (e.g., at least 1.5 hours), to the top of the rankings. In some examples, if a physician is within a last threshold amount of time of their current shift (e.g., 1 hour, 30 minutes), regardless of CI, is moved to the top of the ranking. In some examples, any physician that has been sent a case alert but has not responded is moved to the bottom of the rankings. In some examples, any physician that has not been sent a case alert in a minimum amount of time (e.g., an hour) is moved ahead in the rankings of other physicians that have received a case alert within the minimum amount of time. In some examples, the above rules are applied in the order given above. In some examples, the above rules are applied in a different order. In some examples, only a subset of the above rules are applied.

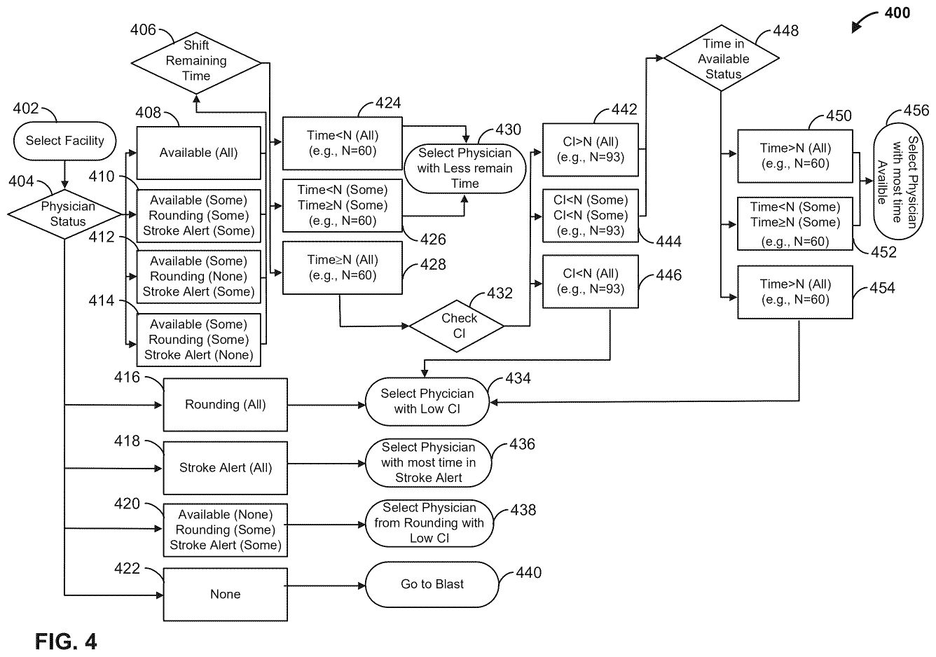

FIG. 4 is a flowchart of an example method 400 to select a physician for an incoming case alert from a facility, and that can be carried out by the telecare computing device 102 of FIG. 1. Although discussed with respect to treatment of strokes, the methods and systems described herein can also be used for other medical issues, such as heart attacks. Upon receiving a case alert from a facility, such as case alert 302, at block 402 a facility is selected. The selected facility corresponds to the facility sending the case alert (e.g., medical facility 111A). At block 404, physician status for a plurality of physicians is determined. The status of each of the plurality of physicians is based at least partially on availability schedule 366 for each of the physicians. Each of the plurality of physicians may be in one of the following status categories: “Available,” “Rounding,” or currently on an alert, such as a “Stroke Alert.” A status of “Available” indicates the physician is currently available for consult. A status of “Stroke Alert” means the physician is currently working on a case alert for a stroke. As such, “Stroke Alert” can mean that the physician is not available. “Rounding” status means a physician is performing rounds at medical facilities, such as nuerohospitalist rounds. For example, physicians in a “Rounding” status may be scheduled separately. Assuming a number of case alerts are received for a medical condition, such as strokes, and there are no available physicians that can consult for strokes (e.g., not qualified, or not available), then “Rounding” physicians may be pulled from their rounds to take stroke consults.

In some examples, all physicians may have a current status of “Available,” as indicated by block 408. In some examples, some physicians (e.g., a first subset of the physicians, including one or more physicians) may have a current status of “Available,” some physicians (e.g., a second subset of the physicians, including one or more physicians) may have a current status of “Rounding,” and some physicians (e.g., a third subset of the physicians, including one or more physicians) may have a current status of “Stroke Alert,” as indicated by block 410. In some examples, some physicians (e.g., a first subset of the physicians) may have a current status of “Available,” some physicians (e.g., a second subset of the physicians) may have a current status of “Stroke Alert,” but no physicians have current a status “Rounding,” as indicated by block 412. In some examples, some physicians (e.g., a first subset of the physicians) may have a current status of “Available,” some physicians (e.g., a second subset of the physicians) may have current a status “Rounding,” but no physicians have a current status of “Stroke Alert,” as indicated by block 414.

In some examples, all physicians may have a current status of “Rounding,” as indicated by block 416. In some examples, all physicians may have a current status of “Stroke Alert,” as indicated by block 418. In some examples, some physicians (e.g., a first subset of the physicians) may have current a status “Rounding,” some physicians (e.g., a second subset of the physicians) may have a current status of “Stroke Alert,” but no physicians have a current status of “Available,” as indicated by block 420. In some examples, there may be no physicians in a current status of “Available,” “Rounding,” or “Stroke Alert,” as indicated by block 422. This may be an example when all physicians are off-duty.

If at least some physicians are “Available,” as indicated by blocks 408, 410, 412, 414, a shift remaining time is determined for each of the physicians at block 406. Telecare computing device 102 may determine the remaining shift time for each physician based on availability schedule 366 for each physician. Availability schedule 366 may identify, for example, a daily shift schedule for each physician, including a time range (e.g., 9 am to 5 pm) of when each physician may be working.

The shift remaining time for each physician may be determined to be above a threshold amount of time (e.g., 60 minutes), or at or below the threshold amount of time. In some examples, all physicians may have remaining shift times that are less than the threshold amount of time, as indicated by block 424. In some examples, some physicians may have remaining shift times that are less than the threshold amount of time, and some physicians may have remaining shift times that are at or above the threshold amount of time, as indicated by block 426. In some examples, all physicians may have remaining shift times that are at or more than the threshold amount of time, as indicated by block 428.

If at least some physicians have remaining shift times that are less than the threshold amount of time, as indicated by blocks 424 and 426, at block 430 the physician with the least remaining shift time is selected. Telecare computing device 102 may generate and transmit a case request, such as case request 304A, to the physician computing device 112A of the selected physician.

If, however, all physicians may have remaining shift times that are at or more than the threshold amount of time, as indicated by block 428, at block 432 the CI for each physician is determined. In some examples, telecare computing device 102 executes physician credentialing algorithm 370 to determine the CIs. In some examples, telecare computing device 102 obtains the CI for each physician from database 116 (e.g., credentialing index 368), which was previously computed. For example, telecare computing device 102 may compute the CI for each physician occasionally, such as monthly, weekly, or nightly, and store the computed Cis in database 116.

Telecare computing device 102 further determines, for each of the physicians, whether their CI exceeds a minimum CI threshold (e.g., 93). For example, telecare computing device 102 may determine if each physician's CI is below, or at or above, the minimum CI threshold. In some examples, the CI of all physicians is at or above the minimum CI threshold, as indicated by block 442. In some examples, the CI index for some of the physicians is at or above the minimum CI threshold, and the CI index for some of the physicians is below the minimum CI threshold, as indicated by block 444. In some examples, the CI of all physicians is below the minimum CI threshold, as indicated by block 446.

If no physician has a CI index at or above the minimum CI threshold, as indicated by block 446, the physician with the lowest CI is selected at block 434. Telecare computing device 102 may generate and transmit a case request, such as case request 304A, to the physician computing device 112A of the selected physician.

If, however, at least some (e.g., at least one) of the physicians have a CI index at or above the minimum CI threshold, as indicated by blocks 442, 444, a “time in available status” is determined for those physicians at block 448. The “time in available status” may be a continuous amount of time each of the physicians has been in “Available” status (e.g., without being assigned a case alert). Telecare computing device 102 may determine the “time in available status” for each of the physicians based on when the last case alert was assigned to the physician since the physician began the current shift. Further, telecare computing device 102 may determine whether each of the physician's “time in available status” is beyond a minimum available status threshold (e.g., 1 hour, 30 minutes).

In some examples, telecare computing device 102 determines that all of the remaining physicians have a computed “time in available status” at or above the minimum available status threshold, as indicated by block 450. In some examples, telecare computing device 102 determines that some of the remaining physicians have a computed “time in available status” at or above the minimum available status threshold, and some of the remaining physicians have a computed “time in available status” below the minimum available status threshold, as indicated by block 452. In some examples, telecare computing device 102 determines that all of the remaining physicians have a computed “time in available status” below the minimum available status threshold, as indicated by block 454.

If at least some (e.g., at least one) physicians have a computed “time in available status” at or above the minimum available status threshold, as indicated by blocks 450, 452, the physician with the greatest “time in available status” can be selected. Otherwise, if no physician has a computed “time in available status” at or above the minimum available status threshold, as indicated by block 454, the physician with the lowest CI can be selected at block 434. Telecare computing device 102 may generate and transmit a case request, such as case request 304A, to the physician computing device 112A of the selected physician.

Back at block 404, if all physicians have a status of “Rounding,” as indicated by block 416, the physician with the lowest CI is selected at block 434. If all physicians have a current status of “Stroke Alert,” as indicated by block 418, the physician with the most amount of time in “Stroke Alert” (e.g., the one likely to be closest to being available) is selected at block 436. For example, telecare computing device 102 may determine the amount of time each of the physicians has been in a current status of “Stroke Alert,” and select the physician that has been in “Stroke Alert” status the greatest amount of time. If some physicians have current a status “Rounding,” some physicians have a current status of “Stroke Alert,” but no physicians have a current status of “Available,” as indicated by block 420, telecare computing device 102 can select the physician that is in a status of “Rounding” with the lowest CI. Telecare computing device 102 may generate and transmit a case request, such as case request 304A, to the physician computing device 112A of the selected physician.

If there are no physicians in a current status of “Available,” “Rounding,” or “Stroke Alert,” as indicated by block 422, telecare computing device 102 will enter a “Blast” mode, as indicated by block 440. In blast mode, telecare computing device 102 transmits a case request 304A to a plurality physicians, and selects the physician that is first to accept the case request 304A (e.g., via a case response 306A). For example, when in “Blast” mode, telecare computing device 102 may generate a secure text or chat message, and may transmit the secure text or chat message to a physician computing device 112A of each of the plurality of physicians.

Each of the thresholds discussed herein may be predetermined, and preconfigured by a user (e.g., via a configuration webpage using I/O device 203 to enter input data). Moreover, in the event that two or more physicians meet a selection criteria, telecare computing device 102 may randomly select one of those physicians, or may select one of the physicians based on a default rule (e.g., alphabetical order, the most number of cases handled over a previous period of time, the least number of cases handled over the previous period of time, an overtime list, a preferred list, etc.).

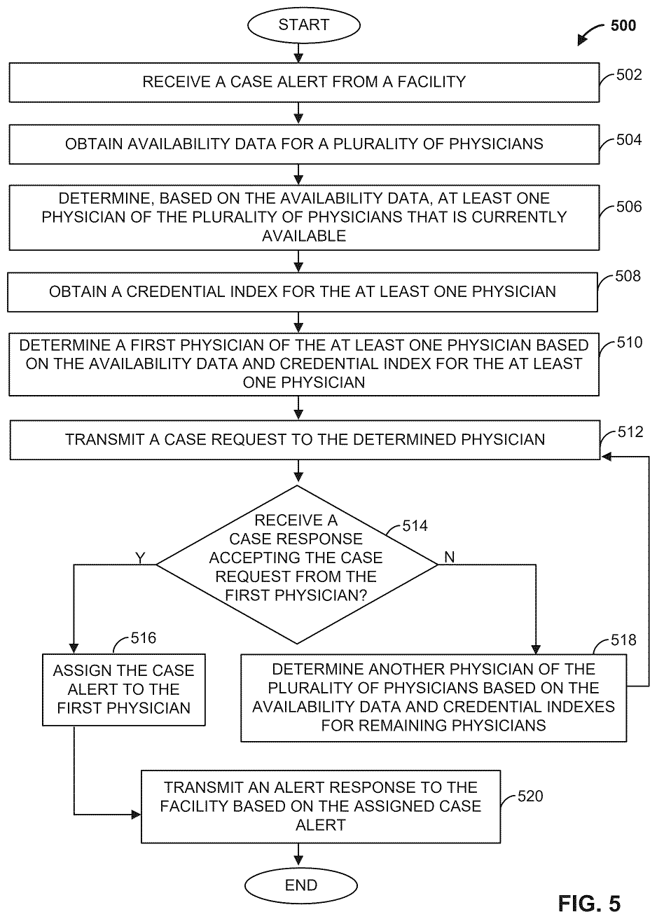

FIG. 5 is a flowchart of an example method 500 that can be carried out by the telecare computing device 102 of FIG. 1. Beginning at step 502, a case alert is received from a facility, such as case alert 302 from facility servers 120A. At step 504, availability data for a plurality of physicians is obtained. For example, telecare computing device 102 may obtain availability schedule 366 for the plurality of physicians from database 116.

Proceeding to step 506, at least one physician that is currently available is determined based on the availability data for the plurality of physicians. For example, telecare computing device 102 may determine all physicians that are currently available, with at least more than a threshold amount of time (e.g., 15 minutes, 30 minutes, 1 hour) remaining in their current shift. At step 508, a credential index is obtained for each of the physicians that are currently available. For example, telecare computing device 102 may obtain physician credentialing algorithm 370 from database 116, and apply physician credentialing algorithm 370 to facility data 350 and/or physician data 360 to determine each physician's credentialing index. In some examples, telecare computing device 102 predetermines the credentialing indexes and stores them in database 116. Telecare computing device 102 may obtain the stored credentialing indexes from database 116.

At step 510, a first physician of the at least one physicians is determined based on the availability data and the credential index for each physician. For example, telecare computing device 102 may obtain physician selection algorithm 380 from database 116, and apply physician selection algorithm 380 to availability schedule 366 and the credentialing indexes for each of the at least one physicians to determine the first physician. At step 512, a case request, such as case request 304A, is transmitted to the physician computing device, such as physician computing device 112A, of the first physician.

Proceeding to step 514, a determination is made as to whether a case response, such as case response 306A, is received from the physician computing device of the first physician. If a case response is received and accepts the case assignment, the method proceeds to step 516. At step 516, the case is assigned to the first physician. For example, telecare computing device 102 may store assignment data in database 116 confirming the assignment. The method then proceeds to step 520, where an alert response, such as alert response 312, is transmitted to the facility in response to the case alert received at step 502.

If, however, at step 514, a case response is received declining the case assignment, the method proceeds to step 518. In addition, at step 514, if a case response is not received within a threshold amount of time (e.g., 2 minutes, 5 minutes), the method also proceeds to step 518. At step 518, another physician is determined. The determination is made based on the availability data and the credential indexes for the remaining physicians (e.g., all available physicians, minus any physicians who were sent a case alert but failed to successfully accept the assignment). The method then proceeds back to step 512, where a case alert is generated and transmitted to the physician computing device 112B of the newly determined physician. The method then ends.

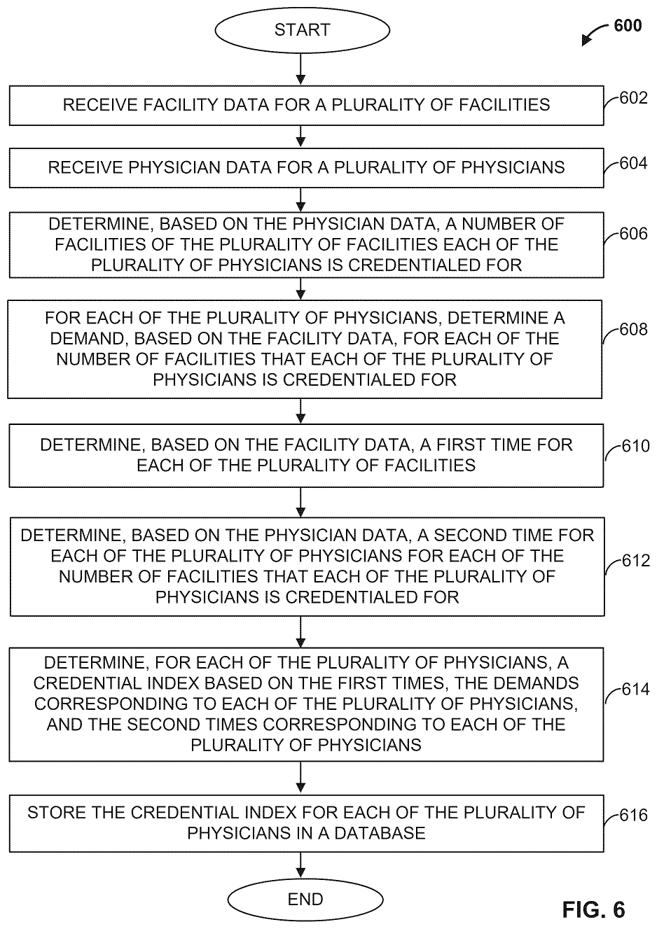

FIG. 6 is a flowchart of an example method 600 that can be carried out by the telecare computing device 102 of FIG. 1. Beginning at step 602, facility data for a plurality of facilities are received. For example, telecare computing device 102 may obtain facility data 350, for each of the plurality of facilities, such as medical facilities 111A, 111B, 111C, from database 116. At step 604, physician data is received for a plurality of physicians. For example, telecare computing device 102 may obtain physician data 360, for each of the plurality of physicians, such as physicians 110A, 110B, 110C, from database 116.

Proceeding to step 606, a number of facilities of the plurality of facilities that each physician is credentialed for is determined based on the physician data. For example, telecare computing device 102 may obtain facility credentials 364 for each physician from database 116, and may determine, based on the facility credentials 364, which facilities of the plurality of facilities the physician is credentialed for (e.g., telecare computing device 102 may compare each of the facilities identified by facility credentials 364 to each of the plurality of facilities to determine if there is a match).

At step 608, for each of the plurality of physicians, a demand is determined for each of the number of facilities that each physician is credentialed for. The demand may be a predicted demand, such as forecasted demand 354. At step 610, a first time is determined for each of the plurality of facilities based on the facility data corresponding to each facility. The first time may be, for example, a video time (e.g., video times per alert type 356) or, in some examples, a total time (e.g., total times per alert type 357). At step 612, a second time is determined for each of the plurality of physicians for each of the number of facilities that each physician is credentialed for. The second times are determined based on the physician data (e.g., video times per case type per facility 362) corresponding to each physician. The second time may be, for example, a video time for the physician (e.g., video times per case type per facility 362) or, in some examples, a total time for the physician (e.g., total times per case type per facility 363).

Proceeding to step 614, for each of the plurality of physicians, a credential index is determined based on the corresponding first times), the demands, and the second times corresponding to each of the plurality of physicians. At step 616, the credential index for each of the plurality of physicians are stored in a database, such as database 116. For example, telecare computing device 102 may generate a ranking of the physicians for each facility based on the credentialing indexes, and store the rankings as physician rankings 358. The method then ends.

In some examples, a system comprises a database (e.g., database 116) and a first computing device (e.g., telecare computing device 102). The first computing device is communicatively coupled to the database. Further, the first computing device is configured to receive a case alert for a case for a facility, and determine a plurality of physicians credentialed at the facility based on physician data stored in the database. The first computing device is also configured to determine an availability status of each of the plurality of physicians based on an availability schedule stored in the database. Further, the first computing device is configured to determine a credentialing index for each of the plurality of physicians, where each credentialing index is based on a forecasted demand of each of a plurality of facilities that each of the plurality of physicians is credentialed for. The first computing device is also configured to determine a first physician of the plurality of physicians based on the availability status and the credentialing index of each of the plurality of physicians. The first computing device is further configured to generate a first case request for the case for the first physician. The first computing device is also configured to transmit the first case request to a second computing device (e.g., physician computing device 112A). The second computing device may be a device of the first physician, for example.

In some examples, the first computing device is configured to receive a case response from the second computing device, and determine that the first physician accepts the case based on the case response. The first computing device is also configured to assign the case to the first physician based on the determination.

In some examples, the first computing device is configured to receive a case response from the second computing device, and to determine that the first physician declines the case based on the case response. The first computing device is also configured to determine a second physician of the plurality of physicians based on the availability status and the credentialing index of each of the plurality of physicians. Further, the first computing device is configured to generate a second case request for the case for the second physician. The first computing device is also configured to transmit the second case request to a third computing device (e.g., physician computing device 112B).

In some examples, the first computing device is configured to generate the credentialing index for each of the plurality of physicians based on the forecasted demand of the facility, a historical demand of the facility, and a video time of the facility.

In some examples, a method by a computing device includes receiving a case alert for a case for a facility, and determining a plurality of physicians credentialed at the facility based on physician data stored in a database. The method also includes determining an availability status of each of the plurality of physicians based on an availability schedule stored in the database. Further, the method may include determining a credentialing index for each of the plurality of physicians, where each credentialing index is based on a forecasted demand of each of a plurality of facilities that each of the plurality of physicians is credentialed for. The method further includes determining a first physician of the plurality of physicians based on the availability status and the credentialing index of each of the plurality of physicians. The method also includes generating a first case request for the case for the first physician. The method further includes transmitting the first case request to a second computing device.

In some examples, the method includes receiving a case response from the second computing device, and determining that the first physician accepts the case based on the case response. The method also includes assigning the case to the first physician based on the determination.

In some examples, the method includes receiving a case response from the second computing device, and determining that the first physician declines the case based on the case response. The method also includes determining a second physician of the plurality of physicians based on the availability status and the credentialing index of each of the plurality of physicians. Further, the method includes generating a second case request for the case for the second physician. The method also includes transmitting the second case request to a third computing device.

In some examples, the method includes generating the credentialing index for each of the plurality of physicians based on the forecasted demand of the facility, a historical demand of the facility, and a video time of the facility.

In some examples, non-transitory computer readable medium has instructions stored thereon, where the instructions, when executed by at least one processor, cause a device to perform operations that include receiving a case alert for a case for a facility, and determining a plurality of physicians credentialed at the facility based on physician data stored in a database. The operations also include determining an availability status of each of the plurality of physicians based on an availability schedule stored in the database. Further, the operations include determining a credentialing index for each of the plurality of physicians, where each credentialing index is based on a forecasted demand of each of a plurality of facilities that each of the plurality of physicians is credentialed for. The operations also include determining a first physician of the plurality of physicians based on the availability status and the credentialing index of each of the plurality of physicians. Further, the operations include generating a first case request for the case for the first physician. The operations also include transmitting the first case request to a second computing device.

In some examples, the operations include receiving a case response from the second computing device, and determining that the first physician accepts the case based on the case response. The operations may also include assigning the case to the first physician based on the determination.

In some examples, the operations include receiving a case response from the second computing device, and determining that the first physician declines the case based on the case response. The operations may also include determining a second physician of the plurality of physicians based on the availability status and the credentialing index of each of the plurality of physicians. Further, the operations may include generating a second case request for the case for the second physician. The operations may also include transmitting the second case request to a third computing device.

In some examples, the operations may include generating the credentialing index for each of the plurality of physicians based on the forecasted demand of the facility, a historical demand of the facility, and a video time of the facility.

Among other advantages, the embodiments described herein may allow for the providing of consultation services for medical diagnosis and treatment in a more quick and efficient manner than conventional systems. The embodiments may reduce lag times, or wait times, when requesting consultations from physicians. For example, the embodiments may avoid a situation where a medical emergency requires consultation from a physician specializing in strokes (e.g., a neurologist), but the physician cannot be found or is otherwise unavailable. The embodiments may also allow multiple medical facilities to request and receive timely consultation services (e.g., within maximum response times) from a select group of highly qualified medical professionals. Moreover, the embodiments may automate the assignment of critical cases to a physician for timely attention, and may further ensure that consults are completed within designated service level agreements or time targets (e.g., maximum case completion times), for example. Persons of ordinary skill in the art having the benefit of these disclosures may recognize other advantages as well.

Although the methods described above are with reference to the illustrated flowcharts, it will be appreciated that many other ways of performing the acts associated with the methods can be used. For example, the order of some operations may be changed, and some of the operations described may be optional.

In addition, the methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A system comprising:
a database; and
a first computing device communicatively coupled to the database, wherein the first computing device is configured to:
receive a case alert for a case for a facility;
determine a plurality of physicians credentialed at the facility based on physician data stored in the database;
determine an availability status of each of the plurality of physicians based on an availability schedule stored in the database;
determine a credentialing index for each of the plurality of physicians, wherein each credentialing index is based on a forecasted demand of each of a plurality of facilities that each of the plurality of physicians is credentialed for;
determine a first physician of the plurality of physicians based on the availability status and the credentialing index of each of the plurality of physicians;
generate a first case request for the case for the first physician; and
transmit the first case request to a second computing device.

2. The system of claim 1, wherein the first computing device is configured to:
receive a case response from the second computing device;
determine that the first physician accepts the case based on the case response; and
assign the case to the first physician based on the determination.

3. The system of claim 1, wherein the first computing device is configured to:
receive a case response from the second computing device;
determine that the first physician declines the case based on the case response;
determine a second physician of the plurality of physicians based on the availability status and the credentialing index of each of the plurality of physicians;
generate a second case request for the case for the second physician; and
transmit the second case request to a third computing device.

4. The system of claim 1, wherein the first computing device is configured to generate the credentialing index for each of the plurality of physicians based on the forecasted demand of the facility, a historical demand of the facility, and a video time of the facility.

5. The system of claim 1, wherein the first computing device is configured to determine a video time for each of the plurality of physicians based on physician data stored in the database, wherein determining the credentialing index for each of the plurality of physicians is based on the corresponding video time.

6. The system of claim 5, wherein the case alert is for a stroke, and the video time for each of the plurality of physicians is a video time for stroke alerts.

7. The system of claim 1, wherein the first computing device is configured to determine a shift remaining time for each of the plurality of physicians based on the on physician data, wherein determining the credentialing index for each of the plurality of physicians is based on the corresponding shift remaining time.

8. The system of claim 7, wherein the first computing device is configured to determine that the shift remaining time for each of the plurality of physicians is beyond a threshold.

9. A method by a first computing device comprising:
receiving a case alert for a case for a facility;
determining a plurality of physicians credentialed at the facility based on physician data stored in a database;
determining an availability status of each of the plurality of physicians based on an availability schedule stored in the database;
determining a credentialing index for each of the plurality of physicians, wherein each credentialing index is based on a forecasted demand of each of a plurality of facilities that each of the plurality of physicians is credentialed for;
determining a first physician of the plurality of physicians based on the availability status and the credentialing index of each of the plurality of physicians;
generating a first case request for the case for the first physician; and
transmitting the first case request to a second computing device.

10. The method of claim 9 comprising:
receiving a case response from the second computing device;
determining that the first physician accepts the case based on the case response; and
assigning the case to the first physician based on the determination.

11. The method of claim 9 comprising:
receiving a case response from the second computing device;
determining that the first physician declines the case based on the case response;
determining a second physician of the plurality of physicians based on the availability status and the credentialing index of each of the plurality of physicians;
generating a second case request for the case for the second physician; and
transmitting the second case request to a third computing device.

12. The method of claim 9 comprising generating the credentialing index for each of the plurality of physicians based on the forecasted demand of the facility, a historical demand of the facility, and a video time of the facility.

13. The method of claim 9 comprising determining a video time for each of the plurality of physicians based on physician data stored in the database, wherein determining the credentialing index for each of the plurality of physicians is based on the corresponding video time.

14. The method of claim 9 comprising determining a shift remaining time for each of the plurality of physicians based on the on physician data, wherein determining the credentialing index for each of the plurality of physicians is based on the corresponding shift remaining time.

15. The method of claim 9 comprising determining that the shift remaining time for each of the plurality of physicians is beyond a threshold.

16. A non-transitory computer readable medium having instructions stored thereon, wherein the instructions, when executed by at least one processor, cause a first computing device to perform operations comprising:
receiving a case alert for a case for a facility;
determining a plurality of physicians credentialed at the facility based on physician data stored in a database;
determining an availability status of each of the plurality of physicians based on an availability schedule stored in the database;
determining a credentialing index for each of the plurality of physicians, wherein each credentialing index is based on a forecasted demand of each of a plurality of facilities that each of the plurality of physicians is credentialed for;
determining a first physician of the plurality of physicians based on the availability status and the credentialing index of each of the plurality of physicians;
generating a first case request for the case for the first physician; and
transmitting the first case request to a second computing device.

17. The non-transitory computer readable medium of claim 16 having instructions stored thereon, wherein the instructions, when executed by the at least one processor, further cause the first computing device to perform operations comprising:
receiving a case response from the second computing device;
determining that the first physician accepts the case based on the case response; and
assigning the case to the first physician based on the determination.

18. The non-transitory computer readable medium of claim 16 having instructions stored thereon, wherein the instructions, when executed by the at least one processor, further cause the first computing device to perform operations comprising:
receiving a case response from the second computing device;
determining that the first physician declines the case based on the case response;
determining a second physician of the plurality of physicians based on the availability status and the credentialing index of each of the plurality of physicians;
generating a second case request for the case for the second physician; and
transmitting the second case request to a third computing device.

19. The non-transitory computer readable medium of claim 16 having instructions stored thereon, wherein the instructions, when executed by the at least one processor, further cause the first computing device to perform operations comprising:
generating the credentialing index for each of the plurality of physicians based on the forecasted demand of the facility, a historical demand of the facility, and a video time of the facility.

20. The non-transitory computer readable medium of claim 16 having instructions stored thereon, wherein the instructions, when executed by the at least one processor, further cause the first computing device to perform operations comprising:
determining a video time for each of the plurality of physicians based on physician data stored in the database, wherein determining the credentialing index for each of the plurality of physicians is based on the corresponding video time.
